SEQUENCE LISTING (20 sequences)

SEQ ID NO: 1 - protein, 1198 aa, Betacoronavirus Severe acute respiratory syndrome- related coronavirus
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 2 - protein, 1297 aa, Betacoronavirus Middle east respiratory syndrome-related coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPW

SEQ ID NO: 3 - protein, 1195 aa, Betacoronavirus Severe acute respiratory syndrome- related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWP

SEQ ID NO: 4 - protein, 419 aa, Betacoronavirus Severe acute respiratory syndrome- related coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 413 aa, Betacoronavirus Middle east respiratory syndrome-related coronavirus
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 6 - protein, 422 aa, Betacoronavirus Severe acute respiratory syndrome- related coronavirus
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 7 - protein, 670 aa, Betacoronavirus Severe acute respiratory syndrome- related coronavirus
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 8 - protein, 725 aa, Betacoronavirus Middle east respiratory syndrome-related coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVE

SEQ ID NO: 9 - protein, 667 aa, Betacoronavirus Severe acute respiratory syndrome- related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLR

SEQ ID NO: 10 - protein, 1295 aa, Betacoronavirus Human coronavirus HKU1
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRRKRRGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDNLEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMYVKWP

SEQ ID NO: 11 - protein, 1100 aa, Alphacoronavirus Human coronavirus 229E
CQTTNGTNTSHSVCNGCVGHSENVFAVESGGYIPSNFSFNNWFLLTNTSSVVDGVVRSFQPLLLNCLWSVSGSQFTTGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGDVEAVNFNVTNAATIVCTVALASYADVLVNVSQTAIANIIYCNSVINRLRCDQLSFDVPDGFYSTSPIQPVELPMSIVSLPVYHKHTFIVLHVKFEHQRGPGKCYNCRPSVINITLANFNETKGPLCVDTSHFTTQFVDNVKLARWSASINTGNCPFSFGKVNNFVKFGSVCFSLKDIPGGCAMPIMANLVNHKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPSNWTTSVQVEYLQITSKPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGVADAERMAMYTGSLIGGIALGGLTSAASIPFSLAIQSRLNYVALQTDVLQENQKILAASFNKAMTNIVDAFTGVNDAITQTSQALQTVATALNKIQDVVNQQGNSLNHLTSQLRQNFQAISSSIQAIYDRLDIIQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWW

SEQ ID NO: 12 - protein, 1296 aa, Alphacoronavirus Human coronavirus NL63
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWP

SEQ ID NO: 13 - protein, 1304 aa, Betacoronavirus Betacoronavirus 1
MFLILLISLPTAFAVIGDLNCTLDPRLKGSFNNRDTGPPSISIDTVDVTNGLGTYYVLDRVYLNTTLFLNGYYPTSGSTYRNMALKGTDLLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHYDTGVVSCLYKRNFTYDVNATYLYFHFYQEGGTFYAYFTDTGFVTKFLFNVYLGMALSHYYVMPLTCIRRPKDGFSLEYWVTPLTPRQYLLAFNQDGIIFNAVDCMSDFMSEIKCKTQSIAPPTGVYELNGYTVQPVADVYRRKPDLPNCNIEAWLNDKSVPSPLNWERKTFSNCNFNMSSLMSFIQADSFTCNNIDAAKIYGMCFSSITIDKFAIPNRRKVDLQLGNLGYLQSSNYRIDTTATSCQLYYNLPAANVSVSRFNPSTWNKRFGFIEDSVFVPQPTGVFTNHSVVYAQHCFKAPKNFCPCSSCSCPGKNNGIGTCPAGTNSLTCDNLCTLDPITLKAPDTYKCPQSKSLVGIGEHCSGLAVKSDYCGNNSCTCQPQAFLGWSADSCLQGDKCNIFANFILHDVNNGLTCSTDLQKANTEIELGVCVNYDLYGISGQGIFVEVNATYYNSWQNLLYDSNGNLYGFRDYITNRTFMIHSCYSGRVSAAYHANSSEPALLFRNIKCNYVFNNSLTRQLQPINYSFDSYLGCVVNAYNSTAISVQTCDLTVGSGYCVDYSKNRRSRRAITTGYRFTNFEPFTVNSVNDSLEPVGGLYEIQIPSEFTIGNMEEFIQTSSPKVTIDCAAFVCGDYAACKLQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALHAIQQGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFNYVPTKYVTAKVSPGLCIAGNRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNHSYINLKDIGTYEYYVKWP

SEQ ID NO: 14 - protein, 441 aa, Betacoronavirus Human coronavirus HKU1
MSYTPGHYAGSRSSSGNRSGILKKTSWADQSERNYQTFNRGRKTQPKFTVSTQPQGNTIPHYSWFSGITQFQKGRDFKFSDGQGVPIAFGVPPSEAKGYWYRHSRRSFKTADGQQKQLLPRWYFYYLGTGPYANASYGESLEGVFWVANHQADTSTPSDVSSRDPTTQEAIPTRFPPGTILPQGYYVEGSGRSASNSRPGSRSQSRGPNNRSLSRSNSNFRHSDSIVKPDMADEIANLVLAKLGKDSKPQQVTKQNAKEIRHKILTKPRQKRTPNKHCNVQQCFGKRGPSQNFGNAEMLKLGTNDPQFPILAELAPTPGAFFFGSKLDLVKRDSEADSPVKDVFELHYSGSIRFDSTLPGFETIMKVLEENLNAYVNSNQNTDSDSLSSKPQRKRGVKQLPEQFDSLNLSAGTQHISNDFTPEDHSLLATLDDPYVEDSVA

SEQ ID NO: 15 - protein, 389 aa, Alphacoronavirus Human coronavirus 229E
MATVKWADASEPQRGRQGRIPYSLYSPLLVDSEQPWKVIPRNLVPINKKDKNKLIGYWNVQKRFRTRKGKRVDLSPKLHFYYLGTGPHKDAKFRERVEGVVWVAVDGAKTEPTGYGVRRKNSEPEIPHFNQKLPNGVTVVEEPDSRAPSRSQSRSQSRGRGESKPQSRNPSSDRNHNSQDDIMKAVAAALKSLGFDKPQEKDKKSAKTGTPKPSRNQSPASSQTSAKSLARSQSSETKEQKHEMQKPRWKRQPNDDVTSNVTQCFGPRDLDHNFGSAGVVANGVKAKGYPQFAELVPSTAAMLFDSHIVSKESGNTVVLTFTTRVTVPKDHPHLGKFLEELNAFTREMQQHPLLNPSALEFNPSQTSPATAEPVRDEVSIETDIIDEVN

SEQ ID NO: 16 - protein, 377 aa, Alphacoronavirus Human coronavirus NL63
MASVNWADDRAARKKFPPPSFYMPLLVSSDKAPYRVIPRNLVPIGKGNKDEQIGYWNVQERWRMRRGQRVDLPPKVHFYYLGTGPHKDLKFRQRSDGVVWVAKEGAKTVNTSLGNRKRNQKPLEPKFSIALPPELSVVEFEDRSNNSSRASSRSSTRNNSRDSSRSTSRQQSRTRSDSNQSSSDLVAAVTLALKNLGFDNQSKSPSSSGTSTPKKPNKPLSQPRADKPSQLKKPRWKRVPTREENVIQCFGPRDFNHNMGDSDLVQNGVDAKGFPQLAELIPNQAALFFDSEVSTDEVGDNVQITYTYKMLVAKDNKNLPKFIEQISAFTKPSSIKEMQSQSSHVAQNTVLNASIPESKPLADDDSAIIEIVNEVLH

SEQ ID NO: 17 - protein, 448 aa, Betacoronavirus Betacoronavirus 1
MSFTPGKQSSSRASSGNRSGNGILKWADQSDQVRNVQTRGRRAQPKQTATSQQPSGGNVVPYYSWFSGITQFQKGKEFEFVEGQGPPIAPGVPATEAKGYWYRHNRGSFKTADGNQRQLLPRWYFYYLGTGPHAKDQYGTDIDGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKILNKPRQKRSPNKQCTVQQCFGKRGPNQNFGGGEMLKLGTSDPQFPILAELAPTAGAFFFGSRLELAKVQNLSGNPDEPQKDVYELRYNGAIRFDSTLSGFETIMKVLNENLNAYQQQDGMMNMSPKPQRQRGHKNGQGENDNISVAVPKSRVQQNKSRELTAEDISLLKKMDEPYTEDTSEI

SEQ ID NO: 18 - protein, 529 aa, Alphainfluenzavirus Influenza A virus
MEARLLVLLCAFAATNADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHHQNEQGSGYAADQKSTQNAIDGITNKVNSVIEKMNTQFTAVGKEFNNLERRIENLNKKVDDGFLDIWTYNAELLVLLENERTLDFHDSNVRNLYEKVKSQLKNNAKEIGNGCFEFYHKCDDACMESVRNGTYDYPKYSEESKLNREEIDGVKLESMGVYQ

SEQ ID NO: 19 - protein, 530 aa, Alphainfluenzavirus Influenza A virus
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGVELKSGYKDW

SEQ ID NO: 20 - protein, 556 aa, Betainfluenzavirus Influenza B virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAA

(19) United States
(12) Patent Application Publication
(10) Pub. No.: US 2022/0170923 A1
(43) Pub. Date: Jun. 2, 2022

(54) PROTEIN MICROARRAY, DETECTION METHOD THEREOF AND EVALUATION METHOD THEREOF

(71) Applicant: NATIONAL CHENG KUNG UNIVERSITY, TAINAN CITY (TW)

(72) Inventor: Guan-Da SYU, Tainan City (TW)

(73) Assignee: NATIONAL CHENG KUNG UNIVERSITY, TAINAN CITY (TW)

(21) Appl. No.: 17/140,186

(22) Filed: Jan. 4, 2021

(30) Foreign Application Priority Data
Dec. 1, 2020 (TW) 109142296

(51) Int. Cl. G01N 33/543; G01N 33/569; G01N 33/68

(52) U.S. Cl. CPC G01N 33/54373; G01N 33/6854; G01N 33/56983

(57) ABSTRACT

A protein microarray, a detection method thereof, and an evaluation method thereof are provided. The protein microarray includes a carrier including a protein array block on a surface thereof and at least one protein immobilized on the protein array block. The at least one protein includes a spike protein and a nucleocapsid protein, and can bind to a first antibody in a to-be-tested sample. A method for detecting virus infection or evaluating the ability of a bioagent to block virus infection includes the steps of respectively adding blood, serum, plasma or the bioagent and a second antibody to the protein microarray and detecting an optical signal. The protein microarray can be used to detect a coronavirus and an influenza virus, and achieve the effect of quickly, sensitively, and accurately confirming whether a subject is infected by the virus.

### CROSS REFERENCE TO RELATED APPLICATION

The present application claims priority to Taiwan Patent Application No. 109142296 filed on Dec. 1, 2020 submitted to Intellectual Property Office, Ministry of Economic Affairs, R.O.C., with the invention titled “protein microarray, detection method thereof, use thereof and kit containing the same”, the entire contents of which are hereby incorporated by reference in this application.

### FIELD OF INVENTION

The present disclosure relates to the technical field of microarray, and particularly, to a protein microarray; the present disclosure also relates to the technical field of a detection method, and particularly, to a detection method of the protein microarray; and the present disclosure also relates to the technical field of evaluation method, and particularly, to an evaluation method of the protein microarray.

### BACKGROUND OF INVENTION

The infectious diseases caused by viruses, such as acute respiratory syndrome caused by an influenza virus or a coronavirus, or severe acute respiratory syndrome caused by a severe acute respiratory syndrome coronavirus, have a high degree of infective power, which are mainly spread directly by the droplets generated by sneezing or coughing, or spread indirectly by contacting contaminants infected by the virus and then touching the mouth or nose. Therefore, if patients infected with an influenza virus or a coronavirus are not accurately tested, the epidemic of infectious diseases will expand quickly and further spread to the world.

For example, a cluster infection of severe special infectious pneumonia was first reported in Wuhan City, mainland China in December 2019, and then it quickly spread in other provinces and cities in mainland China and further spread all over the world. So far, there are more than 50 million confirmed cases worldwide, and more than 1 million deaths. The severe special infectious pneumonia is also known as “coronavirus disease 2019” (COVID-19), which is caused by a severe acute respiratory syndrome coronavirus 2 (SARS-CoV-2). Since the COVID-19 epidemic has spread around the world, and the epidemic is still severe at present, in order to stop the global spread of the COVID-19 epidemic, testing for COVID-19 is a top priority for people suspected of suffering from COVID-19 who have a history of contact with patients suffering from COVID-19 and have upper respiratory symptoms.

At present, the common method for rapid detection of COVID-19 is to collect samples of the nasopharyngeal mucosa from a subject with a nasal swab or a throat swab, and SARS-CoV-2 ribonucleic acids (SARS-CoV-2 RNA) in the nasal swab or the throat swab are detected by real-time polymerase chain reaction (real-time PCR). However, collecting samples from the subject with the nasal swab or the throat swab often causes discomfort in the subject's nasal cavity or throat, thereby causing the subject to sneeze or cough, and further increasing the risk of SARS-CoV-2 spread and the risk of human-to-human transmission. In addition, the sensitivity of real-time PCR for first detecting SARS-CoV-2 RNA in the nasopharyngeal mucosa of the subject is only 59%, and it is necessary to collect samples and test for 3 times to determine whether the subject suffers from COVID-19.

Furthermore, for patients suffering from COVID-19, a bioagent that can effectively treat the patients suffering from COVID-19 has not yet developed around the world.

Therefore, using real-time PCR to detect whether the subject is infected by an influenza virus or a coronavirus has the disadvantages of increasing the spread risk of virus, low sensitivity of the first detection, and the need for multiple tests to confirm the results. Moreover, the bioagent that can effectively block COVID-19 infection has not been successfully developed.

As such, the development of a kit with high sensitivity for rapid detection of influenza virus infection or coronavirus infection and evaluation of the ability of bioagent to block virus infection, and a method for detecting virus infection are problems remained to be resolved in the present technical field.

### SUMMARY OF INVENTION

To solve the problems mentioned above, one object of the present disclosure is to provide a protein microarray. The object of quickly and accurately detecting virus infection may be achieved by immobilizing a spike protein and a nucleocapsid protein from a severe acute respiratory syndrome coronavirus 2 (SARS-CoV-2), also known as COVID-19, on a substrate of the protein microarray.

Another object of the present disclosure is to provide a method for in vitro detection of virus infection in a to-be-tested sample. The object of quickly and accurately detecting whether a subject is infected by a coronavirus or an influenza virus may be achieved by reacting a blood sample, a serum sample or a plasma sample of the subject with the protein microarray.

Still another object of the present disclosure is to provide a method for evaluating the ability of a bioagent to block virus infection. The objects of confirming whether a subject is infected by a coronavirus or an influenza virus or evaluating the ability of a bioagent, such as a monoclonal antibody drug or a receptor blocker to block the virus infection, may be achieved by using the protein microarray for detection.

Still yet another object of the present disclosure is to provide a kit containing the protein microarray. The object of quickly and accurately detecting virus infection may be achieved by use of the protein microarray and a fluorescently labeled antibody or an enzyme-labeled antibody.

In order to achieve the objects mentioned above, the present disclosure provides a protein microarray. The protein microarray may comprise a substrate and at least one protein. A surface of the substrate comprises a plurality of protein array blocks and the at least one protein is immobilized on each of the plurality of protein array blocks. The at least one protein is derived from a virus and comprises a spike protein and a nucleocapsid protein. The at least one protein may comprise an amino acid sequence of SEQ ID NO: 1 and an amino acid sequence of SEQ ID NO: 4, and the at least one protein may specifically bind to a first antibody in a to-be-tested sample or a bioagent.

In one embodiment, the substrate may be a glass slide or a nylon film substrate.

In one embodiment, the surface of the substrate may comprise an aldehyde modified layer or an amino modified layer.

In one embodiment, the at least one protein may further comprise a S1 domain of the spike protein, a hemagglutinin protein or a combination thereof.

In one embodiment, the at least one protein may further comprise an amino acid sequence selected from the group consisting of SEQ ID NO: 2, SEQ ID NO: 3, SEQ ID NO: 5, SEQ ID NO: 6, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In one embodiment, the at least one protein may further comprise an amino acid sequence selected from the group consisting of SEQ ID NO: 7, SEQ ID NO: 8, SEQ ID NO: 9, SEQ ID NO: 18, SEQ ID NO: 19, and SEQ ID NO: 20.

In one embodiment, the virus is selected from the group consisting of severe acute respiratory syndrome coronavirus 2 (SARS-CoV-2), middle east respiratory syndrome coronavirus (MERS-CoV), severe acute respiratory syndrome coronavirus (SARSC-CoV), human coronavirus HKU (HKU-CoV), human coronavirus 229E (229E-CoV), human coronavirus NL63 (NL63-CoV), human coronavirus OC43 (OC43-CoV), influenza A virus subtype H1N1, influenza A virus subtype H3N2, and influenza B virus.

In one embodiment, the to-be-tested sample may be a blood sample, a serum sample or a plasma sample from a subject.

In one embodiment, the first antibody may be a human immunoglobulin G (IgG), a human immunoglobulin A (IgA) or human immunoglobulin M (IgM).

In one embodiment, the bioagent may be a monoclonal antibody drug or a receptor blocker.

In one embodiment, the monoclonal antibody drug may be a murine-derived monoclonal antibody or a rabbit-derived monoclonal antibody.

In one embodiment, the monoclonal antibody drug may be a monoclonal antibody against the spike protein of the SARS-CoV-2, a monoclonal antibody against the S1 domain of the spike protein of the SARS-CoV-2 or a monoclonal antibody against the nucleocapsid protein of the SARS-CoV-2.

In one embodiment, the receptor blocker may be a human angiotensin-converting enzyme 2 on a cell surface.

In order to achieve the objects mentioned above, the present disclosure further provides a method for in vitro detection of virus infection in a to-be-tested sample. The method may comprise steps of:

providing the protein microarray mentioned above;

adding a non-protein blocking reagent to the plurality of protein array blocks of the protein microarray, and reacting for 5 to 10 minutes to obtain a first protein microarray;

providing a to-be-tested sample from a subject, adding the to-be-tested sample to the first protein microarray, and reacting for 50 to 70 minutes followed by washing to obtain a second protein microarray;

providing a second antibody which is fluorescently labeled or enzyme-labeled, adding the second antibody to the second protein microarray, and reacting for 25 to 35 minutes followed by washing to obtain a third protein microarray; and reading an optical signal generated from the third protein microarray by a signal reader.

In one embodiment, after reading the optical signal generated from the third protein microarray by the signal reader, the method further comprises a step of determining whether the subject is infected by a virus, wherein the virus is selected from the group consisting of severe acute respiratory syndrome coronavirus 2 (SARS-CoV-2), middle east respiratory syndrome coronavirus (MERS-CoV), severe acute respiratory syndrome coronavirus (SARSC-CoV), human coronavirus HKU (HKU-CoV), human coronavirus 229E (229E-CoV), human coronavirus NL63 (NL63-CoV), human coronavirus OC43 (OC43-CoV), influenza A virus subtype H1N1, influenza A virus subtype H3N2, and influenza B virus.

In order to achieve the objects mentioned above, the present disclosure further provides a method for evaluating the ability of a bioagent to block virus infection. The method may comprise steps of:

providing the protein microarray mentioned above, adding a non-protein blocking reagent to the plurality of protein array blocks of the protein microarray, and reacting for 5 to 10 minutes to obtain a fourth protein microarray;

providing a bioagent, adding the bioagent to the fourth protein microarray, and reacting for 50 to 70 minutes followed by washing to obtain a fifth protein microarray;

providing a second antibody, adding the second antibody to the fifth protein microarray, and reacting for 25 to 35 minutes followed by washing to obtain a sixth protein microarray, wherein the second antibody is fluorescently labeled or enzyme-labeled; and reading an optical signal generated from the sixth protein microarray by a signal reader.

In one embodiment, after reading the optical signal generated from the third protein microarray by the signal reader, the method further comprises a step of evaluating the ability of the bioagent to block virus infection, wherein the virus is selected from the group consisting of severe acute respiratory syndrome coronavirus 2 (SARS-CoV-2), middle east respiratory syndrome coronavirus (MERS-CoV), severe acute respiratory syndrome coronavirus (SARSC-CoV), human coronavirus HKU (HKU-CoV), human coronavirus 229E (229E-CoV), human coronavirus NL63 (NL63-CoV), human coronavirus OC43 (OC43-CoV), influenza A virus subtype H1N1, influenza A virus subtype H3N2, and influenza B virus.

In one embodiment, the bioagent may be a monoclonal antibody drug or a receptor blocker.

In one embodiment, the monoclonal antibody drug may be a monoclonal antibody against the spike protein of the SARS-CoV-2, a monoclonal antibody against the S1 domain of the spike protein of the SARS-CoV-2 or a monoclonal antibody against the nucleocapsid protein of the SARS-CoV-2.

In one embodiment, the receptor blocker may be a human angiotensin-converting enzyme 2 on a cell surface.

In order to achieve the objects mentioned above, the present disclosure further provides a kit for detecting virus infection in vitro. The kit may comprise the protein microarray mentioned above and a second antibody. The second antibody may specifically bind to the first antibody or the bioagent.

In one embodiment, the second antibody may be fluorescently labeled or enzyme-labeled.

In one embodiment, a fluorescence used for the fluorescently labeled second antibody may comprise cyanine dye Cy3 and cyanine dye Cy5.

In one embodiment, an enzyme used for the enzyme-labeled second antibody may comprise biotin and digoxigenin.

The protein microarray and the kit for detecting virus infection in vitro of the present disclosure may quickly, sensitively, and accurately confirm whether the subject is infected with the virus, and take only about 150 minutes to complete the test. Moreover, since there is no necessary to collect samples of the nasopharyngeal mucosa from the subject with a nasal swab or a throat swab, discomfort in the subject's nasal cavity or throat may not be caused, thereby not causing the subject to sneeze or cough. Thus, the risk of virus spread may be reduced. In addition, the sensitivity and specificity of detecting IgG; IgA or IgM in the serum of the subject using the protein microarray and the kit for detecting virus infection in vitro of the present disclosure may reach up to 97%. Therefore, compared with the detection method by use of the real-time PCR in the prior art, there is no necessary to collect multiple samples of the nasopharyngeal mucosa from the subject in detection of using the protein microarray and the kit for detecting virus infection in vitro of the present disclosure, thereby reducing the sampling times for the subject and reducing the detection times. Furthermore, the protein microarray and the kit for detecting virus infection in vitro of the present disclosure may be used to confirm whether the subject is infected by different types of viruses, such as a coronavirus and an influenza virus, or to evaluate the ability of the bioagent, such as a monoclonal antibody drug or a receptor blocker, to block virus infection. The types of the viruses may comprise severe acute respiratory syndrome coronavirus 2, middle east respiratory syndrome coronavirus, severe acute respiratory syndrome coronavirus, human coronavirus HKU, human coronavirus 229E, human coronavirus NL63, human coronavirus OC43, influenza A virus subtype H1N1, influenza A virus subtype H3N2, influenza B virus, or any combination thereof.

### BRIEF DESCRIPTION OF DRAWINGS

FIG. 1A is a resultant graph of the binding specificity between a protein microarray of the present disclosure and a human angiotensin-converting enzyme 2 (ACE2) on a cell surface. Data are analyzed by t-test, * p<0.05 and *** p<0.001, compared with MERS-CoV.

FIG. 1B is a resultant graph of the binding specificity between a protein microarray of the present disclosure and a monoclonal antibody against a 51 domain of a spike protein of SARS-CoV-2 (αS protein mAb). Data are analyzed by t-test, * p<0.05 and *** p<0.001, compared with MERS-CoV.

FIG. 1C is a resultant graph of the binding specificity between a protein microarray of the present disclosure and a monoclonal antibody against a nucleocapsid protein of SARS-CoV-2 (αN protein mAb). Data are analyzed by t-test, * p<0.05 and *** p<0.001, compared with MERS-CoV.

FIG. 2A is a resultant graph of the Cy3 background intensity of the protein microarray of the present disclosure after blocking with 5% bovine serum albumin (BSA) for 1 hour or a non-protein reagent (HyBlock) for 10 minutes, respectively. Data are analyzed by t-test, ****p<0.0001, compared with BSA-1 hour.

FIG. 2B is a resultant graph of the Cy5 background intensity of the protein microarray of the present disclosure after blocking with 5% bovine serum albumin (BSA) for 1 hour or a non-protein reagent (HyBlock, Hycell International Co. Ltd., Taiwan) for 10 minutes, respectively. Data are analyzed by t-test, ****p<0.0001, compared with BSA-1 hour.

FIG. 3A is a resultant graph of the serum IgG reactivity to the spike protein of SARS-CoV-2 by use of the protein microarray of the present disclosure for detection. The serum IgG is obtained from a healthy subject or a patient suffering from COVID-19. Data are analyzed by t-test, ****p<0.0001, compared with the healthy subject.

FIG. 3B is a resultant graph of the serum IgG reactivity to the nucleocapsid protein of SARS-CoV-2 by use of the protein microarray of the present disclosure for detection. The serum IgG is obtained from the healthy subject or the patient suffering from COVID-19. Data are analyzed by t-test, ****p<0.0001, compared with the healthy subject.

FIG. 3C is a resultant graph of the serum IgG reactivity to the spike protein of SARS-CoV by use of the protein microarray of the present disclosure for detection. The serum IgG is obtained from the healthy subject or the patient suffering from COVID-19. Data are analyzed by t-test, ****p<0.0001, compared with the healthy subject.

FIG. 3D is a resultant graph of the serum IgG reactivity to the nucleocapsid protein of SARS-CoV by use of the protein microarray of the present disclosure for detection. The serum IgG is obtained from the healthy subject or the patient suffering from COVID-19. Data are analyzed by t-test, ****p<0.0001, compared with the healthy subject.

FIG. 3E is a resultant graph of a serum IgA reactivity to the spike protein of SARS-CoV-2 by use of the protein microarray of the present disclosure for detection. The serum IgA is obtained from the healthy subject or the patient suffering from COVID-19. Data are analyzed by t-test, ****p<0.0001, compared with the healthy subject.

FIG. 3F is a resultant graph of the serum IgA reactivity to the nucleocapsid protein of SARS-CoV-2 by use of the protein microarray of the present disclosure for detection. The serum IgA is obtained from the healthy subject or the patient suffering from COVID-19. Data are analyzed by t-test, ****p<0.0001, compared with the healthy subject.

FIG. 3G is a resultant graph of the serum IgA reactivity to the spike protein of SARS-CoV by use of the protein microarray of the present disclosure for detection. The serum IgA is obtained from the healthy subject or the patient suffering from COVID-19. Data are analyzed by t-test, ****p<0.0001, compared with the healthy subject.

FIG. 3H is a resultant graph of the serum IgA reactivity to the nucleocapsid protein of SARS-CoV by use of the protein microarray of the present disclosure for detection. The serum IgA is obtained from the healthy subject or the patient suffering from COVID-19. Data are analyzed by t-test, ****p<0.0001, compared with the healthy subject.

FIG. 4A is a resultant graph of the serum IgG cross-reactivity to the spike protein of SARS-CoV-2 and the S1 domain of the spike protein of SARS-CoV-2 by use of the protein microarray of the present disclosure for detection. The serum IgG is obtained from the patient suffering from COVID-19. Data are analyzed by t-test, p<0.0001.

FIG. 4B is a resultant graph of the relationship of the serum IgG cross-reactivity to the spike protein of SARS-CoV-2 and the spike protein of SARS-CoV by use of the protein microarray of the present disclosure for detection. The serum IgG is obtained from the patient suffering from COVID-19. Data are analyzed by t-test, p<0.005.

FIG. 4C is a resultant graph of the relationship of the serum IgG cross-reactivity to the spike protein of SARS-CoV-2 and the spike protein of HKU-CoV by use of the protein microarray of the present disclosure for detection. The serum IgG is obtained from the patient suffering from COVID-19. Data are analyzed by t-test, p<0.0001.

FIG. 4D is a resultant graph of the relationship of the serum IgG cross-reactivity to the spike protein of SARS-CoV-2 and the spike protein of OC43-CoV by use of the protein microarray of the present disclosure for detection. The serum IgG is obtained from the patient suffering from COVID-19. Data are analyzed by t-test, p<0.005.

FIG. 4E is a resultant graph of the relationship of the serum IgG cross-reactivity to the spike protein of SARS-CoV-2 and a hemagglutinin protein of influenza A virus subtype H3N2 by use of the protein microarray of the present disclosure for detection. The serum IgG is obtained from the patient suffering from COVID-19. Data are analyzed by t-test, p<0.001.

FIG. 4F is a resultant graph of the relationship of the serum IgG cross-reactivity to the nucleocapsid protein of SARS-CoV-2 and the nucleocapsid protein of SARS-CoV by use of the protein microarray of the present disclosure for detection. The serum IgG is obtained from the patient suffering from COVID-19. Data are analyzed by t-test, p<0.0001.

FIG. 5A is a resultant graph of the serum IgA cross-reactivity to the spike protein of SARS-CoV-2 and the S1 domain of the spike protein of SARS-CoV-2 by use of the protein microarray of the present disclosure for detection. The serum IgA is obtained from the patient suffering from COVID-19. Data are analyzed by t-test, p<0.0001.

FIG. 5B is a resultant graph of the relationship of the serum IgA cross-reactivity to the spike protein of SARS-CoV-2 and the spike protein of SARS-CoV by use of the protein microarray of the present disclosure for detection. The serum IgA is obtained from the patient suffering from COVID-19. Data are analyzed by t-test, p<0.0001.

FIG. 5C is a resultant graph of the relationship of the serum IgA cross-reactivity to the spike protein of SARS-CoV-2 and the spike protein of HKU-CoV by use of the protein microarray of the present disclosure for detection. The serum IgA is obtained from the patient suffering from COVID-19. Data are analyzed by t-test, p<0.0001.

FIG. 5D is a resultant graph of the relationship of the serum IgA cross-reactivity to the spike protein of SARS-CoV-2 and the spike protein of OC43-CoV by use of the protein microarray of the present disclosure for detection. The serum IgA is obtained from the patient suffering from COVID-19. Data are analyzed by t-test, p<0.0001.

FIG. 5E is a resultant graph of the relationship of the serum IgA cross-reactivity to the nucleocapsid protein of SARS-CoV-2 and the nucleocapsid protein of SARS-CoV by use of the protein microarray of the present disclosure for detection. The serum IgA is obtained from the patient suffering from COVID-19. Data are analyzed by t-test, p<0.0001.

FIG. 5F is a resultant graph of the relationship of the serum IgA cross-reactivity to the nucleocapsid protein of SARS-CoV-2 and the spike protein of 229E-CoV by use of the protein microarray of the present disclosure for detection. The serum IgA is obtained from the patient suffering from COVID-19. Data are analyzed by t-test, p<0.001.

### DETAILED DESCRIPTION OF PREFERRED EMBODIMENTS

The following provides specific embodiments to illustrate the implementation of the present disclosure. A person having ordinary skill in the art can understand other advantages and effects of the present disclosure from the contents disclosed in the present specification. However, the exemplary embodiments disclosed in the present disclosure are only for illustrative purposes and should not be regarded as limiting the scope of the present disclosure. In other words, the present disclosure can also be implemented or applied by other different specific embodiments, and various details in the present specification can also be modified and changes based on different viewpoints and applications without departing from the concept of the present disclosure.

Unless otherwise indicated herein, the singular forms “one” and “the” used in the specification and the appended claims of the present disclosure include the plural. Unless otherwise indicated herein, the term “or” used in the specification and the appended claims of the present disclosure includes the meaning of “and/or”.

### Example 1: Preparation of Protein Microarray

The proteins from different viruses with histidine tag (His-tag) listed in Table 1 are purchased from Sino Biological Inc. (mainland China), and each of the proteins is used as a biomarker for detecting virus infection. Spike proteins (hereinafter referred to as “S protein”) shown in SEQ ID NO: 1 ′ SEQ ID NO: 2, and SEQ ID NO: 3 are respectively from severe acute respiratory syndrome coronavirus 2 (SARS-CoV-2), middle east respiratory syndrome coronavirus (MERS-CoV), and severe acute respiratory syndrome coronavirus (SARSC-CoV); nucleocapsid proteins (hereinafter referred to as “N protein”) shown in SEQ ID NO: 4 ′ SEQ ID NO: 5, and SEQ ID NO: 6 are respectively from SARS-CoV-2, MERS-CoV, and SARSC-CoV; and 51 domains of the spike proteins (hereinafter referred to as “51 domain of S protein”) shown in SEQ ID NO: 7 ′ SEQ ID NO: 8, and SEQ ID NO: 9 are respectively from SARS-CoV-2, MERS-CoV, and SARSC-CoV; the S proteins shown in SEQ ID NO: 10 ′ SEQ ID NO: 11, SEQ ID NO: 12, and SEQ ID NO: 13 are respectively from human coronavirus HKU (HKU-CoV), human coronavirus 229E (229E-CoV), human coronavirus NL63 (NL63-CoV), and human coronavirus OC43 (OC43-CoV); the N proteins shown in SEQ ID NO: 14 ′ SEQ ID NO: 15, SEQ ID NO: 16, and SEQ ID NO: 17 are respectively from HKU-CoV, 229E-CoV, NL63-CoV, and OC43-CoV; and hemagglutinin proteins (hereinafter referred to as “HA protein”) shown in SEQ ID NO: 18 ′ SEQ ID NO:19, and SEQ ID NO: 20 are respectively from influenza A virus subtype H1N1, influenza A virus subtype H3N2, and influenza B virus.

The sequence number shown in SEQ ID NO: 1 is renumbered from 1 to 1198 according to Val 16 to Pro1213 of Uniprot ID P0DTC2 sequence. The sequence number shown in SEQ ID NO: 7 is renumbered from 1 to 670 according to Val 16 to Arg685 of Uniprot ID P0DTC2 sequence. The sequence number shown in SEQ ID NO: 11 is renumbered from 1 to 1100 according to Cys16-Trp1115 of Uniprot ID P15423 sequence.

TABLE 1
List of the proteins from different viruses with His-tag

| protein | virus | amino acid | Uniprot ID |
|---|---|---|---|
| S | SARS-CoV-2 | SEQ ID NO: 1 | P0DTC2 (Val 16-Pro1213) |
| protein | MERS-CoV | SEQ ID NO: 2 | K9N5Q8 (Met1-Trp1297) |
| | SARS-CoV | SEQ ID NO: 3 | P59594 (Met1-Pro1195) |
| N | SARS-CoV-2 | SEQ ID NO: 4 | P0DTC9 (Met1-A1a419) |
| protein | MERS-CoV | SEQ ID NO: 5 | R9UM87 (Met1-Asp413) |
| | SARS-CoV | SEQ ID NO: 6 | P59595 (Met1-A1a422) |
| S1 | SARS-CoV-2 | SEQ ID NO: 7 | P0DTC2 (Val 16-Arg685) |
| domain | MERS-CoV | SEQ ID NO: 8 | K9N5Q8 (Met1-G1u725) |
| of S | SARS-CoV | SEQ ID NO: 9 | P59594 (Met1-Arg667) |
| protein | | | |
| S | HKU-CoV | SEQ ID NO: 10 | Q0ZME7 (Met1-Pro1295) |
| protein | 229E-CoV | SEQ ID NO: 11 | P15423 (Cys16-Trp1115) |
| | NL63-CoV | SEQ ID NO: 12 | Q6Q152 (Met1-Pro1296) |
| | OC43-CoV | SEQ ID NO: 13 | P36334 (Met1-Pro1304) |
| N | HKU-CoV | SEQ ID NO: 14 | Q5MQC6 (Met1-Ala441) |
| protein | 229E-CoV | SEQ ID NO: 15 | P15130 (Met1-Asn389) |
| | NL63-CoV | SEQ ID NO: 16 | Q6Q1R8 (Met1-His377) |
| | OC43-CoV | SEQ ID NO: 17 | P33469 (Met1-I1e448) |
| HA | influenza | SEQ ID NO: 18 | Q9WFX3 (Met 1-Gln 529) |
| protein | A virus | | |
| | subtype | | |
| | H1N1 | | |
| | influenza | SEQ ID NO: 19 | P03437 (Met 1-Trp 530) |
| | A virus | | |
| | subtype | | |
| | H3N2 | | |
| | influenza | SEQ ID NO: 20 | P03460 (Met 1-Ala 556) |
| | B virus | | |

After rinsing a glass slide with water, ethanol, acetone and methanol in sequence, wash the glass slide with 20% KOH solution at 65° C. for 2 hours, followed by wishing with H₂SO₄/H₂O₂ with a volume ratio of 3:1 for 12 minutes to obtain a cleaned glass slide. The cleaned glass slide is then coated by the steps of incubating the cleaned glass slide with 2.5% 3-aminopropyl triethoxysilane (dissolved in alcohol) for 5 minutes, then washing and being dried; incubating with 0.5% glutaraldehyde (dissolved in 0.05 M sodium borate pH 8.5) for 16 hours, then washing and being dried to obtain a surface-treated glass slide. The surface-treated glass slide is stored in a sealed vacuum bag at 4° C.

Each surface-treated glass slide has 14 blocks. Triplicates of each of the proteins shown in Table 1 and each of the 11 control group samples shown in Table 2 are printed in each block (9×10 format) on the surface-treated glass slide by a microarray spotter (CapitalBio SmartArrayer™ 136, mainland China) to obtain a protein microarray. The protein microarray is immobilized overnight at room temperature, then vacuum sealed, and stored at 4° C. for short-term (less than 6 months) or at −80° C. for long-term storage (6 months to a few years).

Table 2 List of the control group samples

### Example 2 Analysis of Receptor Specificity and Antibody Specificity of the Protein Microarray

The receptor specificity of the protein microarray is analyzed by a human angiotensin-converting enzyme 2 (ACE2) on a cell surface purchased from Sino Biological Inc. (mainland China). The antibody specificity of the protein microarray is analyzed by antibody drugs, such as a monoclonal antibody against the S1 domain of S protein of SARS-CoV-2 (hereinafter referred to as αS protein mAb) purchased from Sino Biological Inc. (mainland China) and a monoclonal antibody against the N protein of SARS-CoV-2 (hereinafter referred to as αN protein mAb) purchased from Sino Biological Inc. (mainland China).

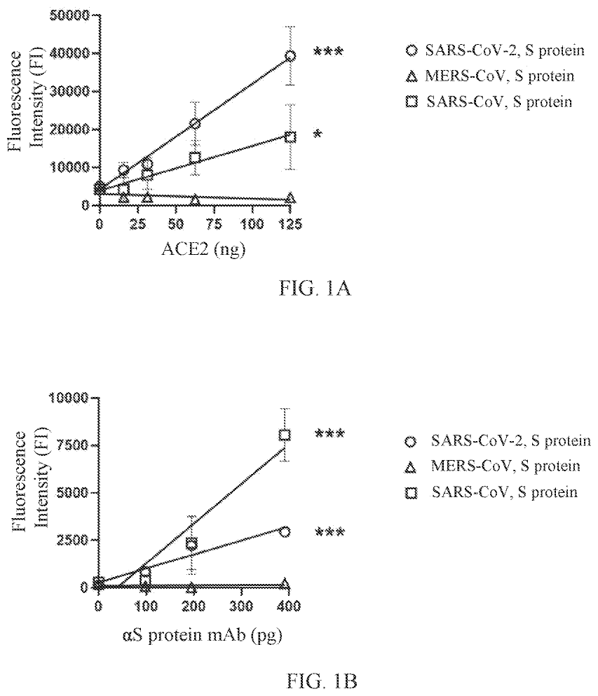

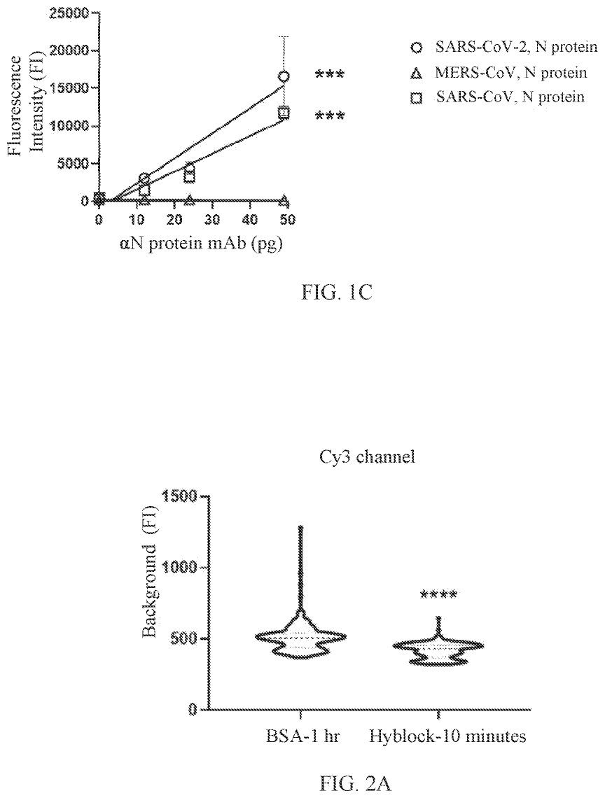

The ACE2 receptors of 25 ng, 50 ng, 75 ng, 100 ng, and 125 ng, which have been serially diluted, are reacted with the protein microarray for 1 hour, and then the protein microarray is reacted with a Cy3 labeled anti-human IgG antibody for 30 minutes. Referring to FIG. 1A, the result shows that nanogram-level of ACE2 may significantly bind to the S protein of SARS-CoV-2 and the S protein of SARS-CoV on the protein microarray but may not bind to the S protein of MERS-CoV.

The αS protein mAb of 100 pg, 200 pg, 300 pg, and 400 pg, which have been serially diluted, are reacted with the protein microarray for 1 hour, and then the protein microarray is reacted with a Cy5 labeled anti-rabbit IgG antibody for 30 minutes. Referring to FIG. 1B, the result shows that picogram-level of αS protein mAb may significantly bind to the S protein of SARS-CoV-2 and the S protein of SARS-CoV on the protein microarray, but may not bind to the S protein of MERS-CoV.

The αN protein mAb of 10 pg, 20 pg, 30 pg, 40 pg, and 50 pg which have been serially diluted, are reacted with the protein microarray for 1 hour, and then the protein microarray is reacted with a Cy5 labeled anti-rabbit IgG antibody for 30 minutes. Referring to FIG. 1C, the result shows that picogram-level of αN protein mAb may significantly bind to the N protein of SARS-CoV-2 and the N protein of SARS-CoV on the protein microarray, but may not bind to the N protein of MERS-CoV.

Based on the above, the results show that the protein microarray may effectively and specifically bind to the ACE2 receptor, the αS protein mAb and the αN protein mAb. Moreover, the minimum detection limits of the αS protein mAb and the αN protein mAb are 200 pg and 10 pg, respectively. Therefore, it is demonstrated that the protein microarray may be used to evaluate the abilities of a ACE2 receptor blocker and a monoclonal antibody drug against a virus to block virus infection.

### Example 3: Analysis of the Reactivity of the Protein Microarray with an Immunoglobulin G (IgG) and an Immunoglobulin a (IgA)

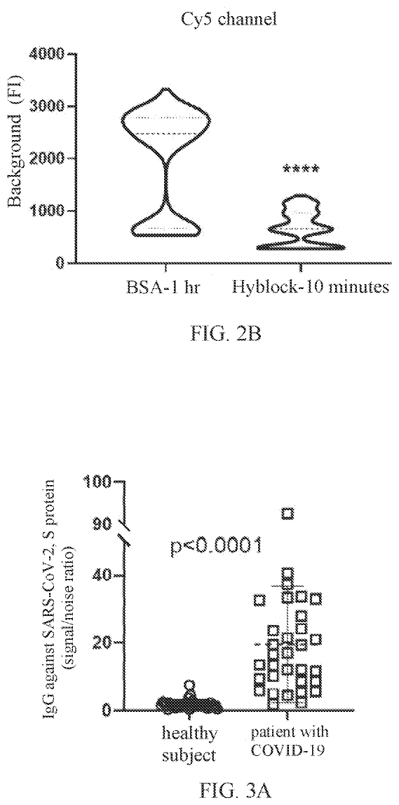

In order to confirm the influence of a blocking buffer on the accuracy of the reading of the protein microarray, each of the protein microarrays is respectively blocked with 5% BSA blocking reagent for 1 hour and HyBlock reagent (purchased from Hycell International Co. Ltd., Taiwan) which is a non-protein blocking reagent for 10 minutes, then each of the protein microarrays is reacted with 0.1 μl serum from COVID-19 patients for 1 hour, followed by washing with Tris-buffered saline with 0.1% Tween® 20 (TBST) buffer, and then each of the protein microarrays is reacted with Cy3 labeled anti-human IgG antibody or Cy5 labeled anti-human IgA antibody for 30 minutes. Finally, each of the protein microarrays is washed with TBST buffer, and then the background fluorescence of each of the protein microarrays is detected by a fluorescence detection system (“Caduceus” SpinScan Microarray Scanner HC-BS01, Caduceus Biotechnology Inc., Taiwan). Referring to FIG. 2A and FIG. 2B, the results show that blocking the non-specific antigen on the protein microarray with the HyBlock reagent shows a cleaner background with superior blocking time, thereby shortening the analysis time compared to 5% BSA blocking reagent.

To profile the reactivities of serum IgG and serum IgA from a subject to each protein on the protein microarray, each of the protein microarrays is blocked with HyBlock reagent for 10 minutes, and then reacted with 0.1 μl of serum from 32 patients suffering from COVID-19 and from 32 healthy subjects for 1 hour, followed by washing with TBST buffer. Finally, each of the protein microarrays is reacted with a Cy3 labeled anti-human IgG antibody or a Cy5 labeled anti-human IgA antibody for 30 minutes, followed by washing with TBST buffer. Cy3 fluorescence or Cy5 fluorescence of each of the protein microarrays is detected by the fluorescence detection system microarray (“Caduceus” SpinScan Microarray Scanner HC-BS01, Caduceus Biotechnology Inc., Taiwan).

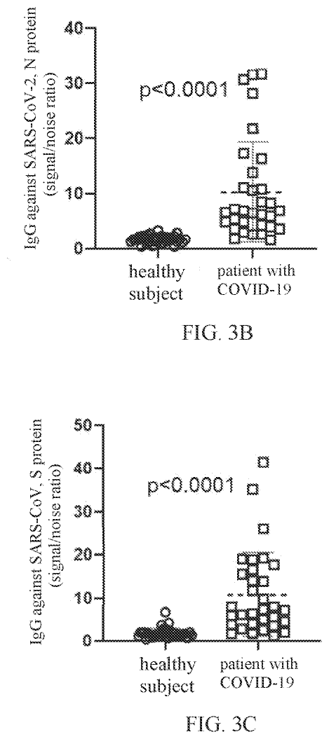

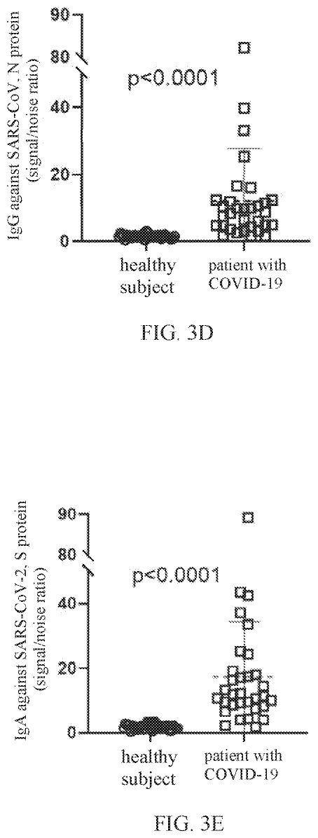

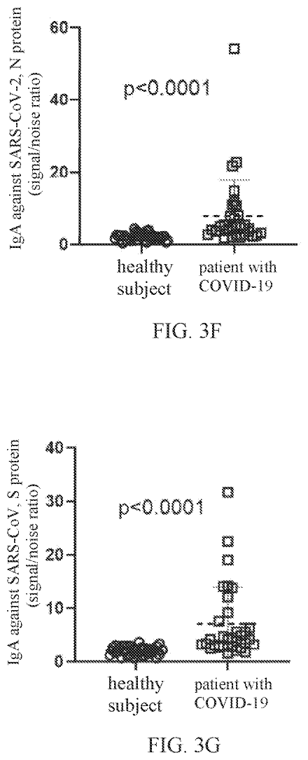

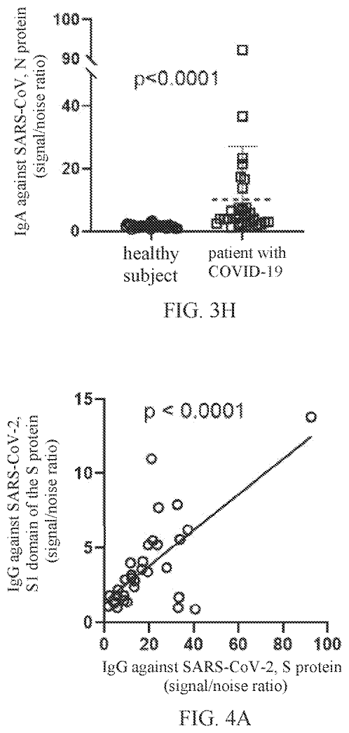

Referring to FIG. 3A and FIG. 3B, the results respectively show that the protein microarray may significantly distinguish the reactivity of the S protein of SARS-CoV-2 to the serum IgG from COVID-19 patients from that of the healthy subjects, and the reactivity of the N protein of SARS-CoV-2 to the serum IgG from COVID-19 patients from that of the healthy subjects. Referring to FIG. 3C and FIG. 3D, the results respectively show that the protein microarray may significantly distinguish the reactivity of the S protein of SARS-CoV to the serum IgG from COVID-19 patients from that of the healthy subjects, and the reactivity of the N protein of SARS-CoV to the serum IgG from COVID-19 patients from that of the healthy subjects.

Referring to FIG. 3E and FIG. 3F, the results respectively show that the protein microarray may significantly distinguish the reactivity of the S protein of SARS-CoV-2 to the serum IgA from COVID-19 patients from that of the healthy subjects, and the reactivity of the N protein of SARS-CoV-2 to the serum IgA from COVID-19 patients from that of the healthy subjects. Referring to FIG. 3G and FIG. 3H, the results respectively show that the protein microarray may significantly distinguish the reactivity of the S protein of SARS-CoV to the serum IgA from COVID-19 patients from that of the healthy subjects, and the reactivity of the N protein of SARS-CoV to the serum IgA from COVID-19 patients from that of the healthy subjects.

Based on the above, it is demonstrated that the protein microarray may significantly distinguish the reactivity of each protein such as the S protein or N protein from SARS-CoV-2 and SARS-CoV to the serum IgG and serum IgA from COVID-19 patients from that of the healthy subjects.

### Example 4: Evaluation of the Specificity and Sensitivity of Protein Microarrays with a Single Biomarker or a Combination of Two Biomarkers for Detecting the Serum IgG or the Serum IgA from the Patients Suffering from COVID-19

The protein microarrays with a single biomarker or a combination of two biomarkers are blocked with HyBlock regent for 10 minutes, and each of the protein microarrays is reacted with 0.1 μl serum of 36 patients suffering from a COVID-19 for 1 hour, followed by washing with TBST buffer, and hybridized with Cy3 labeled anti-human IgG antibody or Cy5 labeled anti-human IgA antibody for 30 minutes. Finally, each of the protein microarrays is reacted with a Cy3 labeled anti-human IgG antibody or a Cy5 labeled anti-human IgA antibody for 30 minutes, followed by washing with TBST buffer. Cy3 fluorescence or Cy5 fluorescence of each of the protein microarrays is detected by the fluorescence detection system microarray (“Caduceus” SpinScan Microarray Scanner HC-BS01, Caduceus Biotechnology Inc., Taiwan).

Referring to Table 4, the results show that the sensitivity and specificity of the protein microarray with a single biomarker of the S protein from SARS-CoV-2 for the detection of serum IgG in patients suffering from COVID-19 are 90.6% and 97.2%, respectively. Moreover, the sensitivity and specificity of the protein microarray with a single biomarker of the S protein from SARS-CoV-2 for detection of serum IgA in patients suffering from COVID-19 are 84.4% and 100%, respectively. Furthermore, the sensitivity and specificity of the protein microarray with a combination of two biomarkers, the S protein of SARS-CoV-2 and the N protein of SARS-CoV-2 for the detection of serum IgG in COVID-19 patients are as high as 97%.

TABLE 4
Results of the specificity and sensitivity of protein microarrays
with a single biomarker or a combination of two biomarkers
for detecting the serum IgG or the serum IgA from the
patients suffering from COVID-19

| Biomarker and serum | sensitivity | specificity |
|---|---|---|
| S protein of SARS-CoV-2, IgG | 90.6 | 97.2 |
| S protein of SARS-CoV-2, IgA | 84.4 | 100 |
| N protein of SARS-CoV-2, IgG | 65.6 | 100 |
| N protein of SARS-CoV, IgG | 65.6 | 100 |
| S protein of SARS-CoV, IgG | 65.6 | 97.2 |
| S protein of SARS-CoV-2, IgG + N protein of | 96.9 | 97.2 |
| SARS-CoV-2, IgG | | |
| S protein of SARS-CoV-2, IgG + S protein of | 93.8 | 97.2 |
| SARS-CoV-2, IgA | | |
| S protein of SARS-CoV-2, IgG + N protein of | 93.8 | 97.2 |
| SARS-CoV, IgG | | |
| S protein of SARS-CoV-2, IgG + S protein of | 90.6 | 97.2 |
| SARS-CoV, IgG | | |

### Example 5 Analysis of the Cross-Reactivity Between the S Protein and the N Protein of SARS-CoV-2 on the Protein Microarray and Different Types of Viruses

The protein microarrays are used to detect the reactivity of the IgG in the serum from the patients suffering from COVID-19 with the S protein of SARS-CoV-2, the S1 domain of S protein of SARS-CoV-2, the S protein of SARS-CoV, the S protein of HKU-CoV, the S protein of OC43-CoV, the HA protein of influenza A H3N2 subtype, the N protein of SARS-CoV-2, and the N protein of SARS-CoV, and further analyze the cross-reactivity of the S protein of SARS-CoV-2 with the S1 domain of S protein of SARS-CoV-2, the S protein of SARS-CoV, the S protein of HKU-CoV, the S protein of OC43-CoV, and the HA protein of influenza A H3N2 subtype, as well as the cross-reactivity between the N protein of SARS-CoV-2 and the N protein of SARS-CoV.

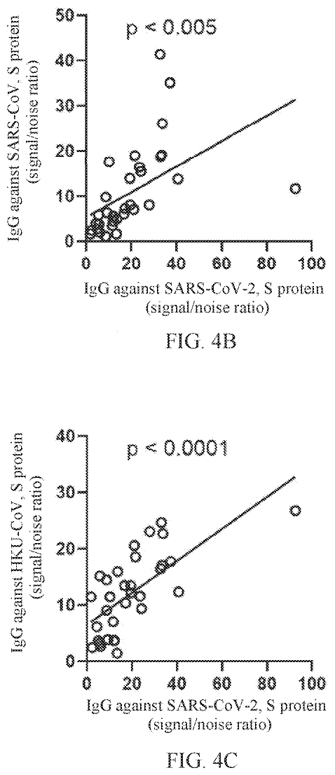

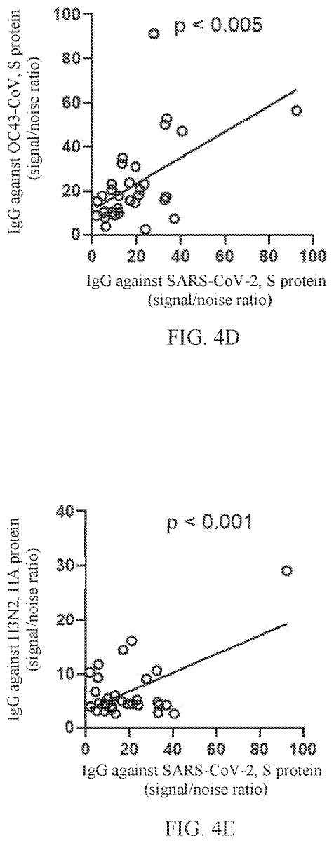

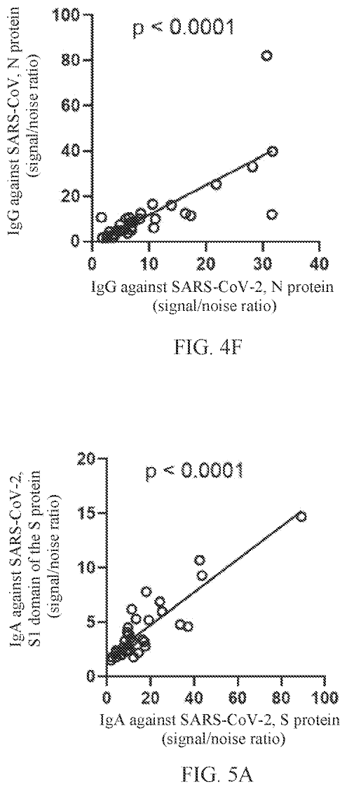

Referring to FIG. 4A to FIG. 4E, the results show, in the serum from the patients suffering from COVID-19, a positive cross-reactive correlation of the IgG against the S protein of SARS-CoV-2 with the IgGs against the S1 domain of S protein of SARS-CoV-2, the S protein of SARS-CoV, the S protein of HKU-CoV, the S protein of OC43-CoV, and the HA protein of influenza A H3N2 subtype. Moreover, as shown in FIG. 4F, the result shows, in the serum from the patients suffering from COVID-19, a positive cross-reactive correlation of the IgG against the N protein of SARS-CoV-2 with the IgG against the N protein of SARS-CoV.

The protein microarrays are used to detect the reactivity of the IgA in the serum from the patients suffering from COVID-19 with the S protein of SARS-CoV-2, the S1 domain of S protein of SARS-CoV-2, the S protein of SARS-CoV, the S protein of HKU-CoV, the S protein of OC43-CoV, the N protein of SARS-CoV-2, the N protein of SARS-CoV, and the S protein of OC43-CoV, and further analyze the cross-reactivity of the S protein of SARS-CoV-2 with the S1 domain of S protein of SARS-CoV-2, the S protein of SARS-CoV, the S protein of HKU-CoV, and the S protein of OC43-CoV, as well as the cross-reactivity between the N protein of SARS-CoV-2 and the N protein of SARS-CoV, and the S protein of 229E-CoV.

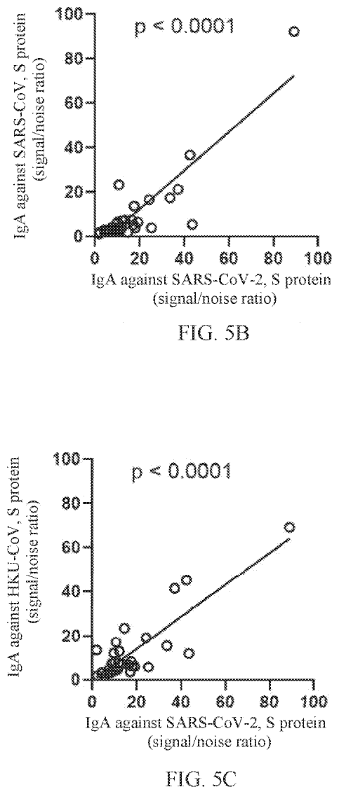

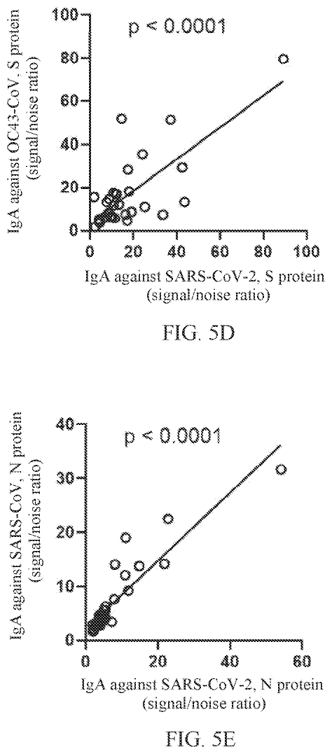

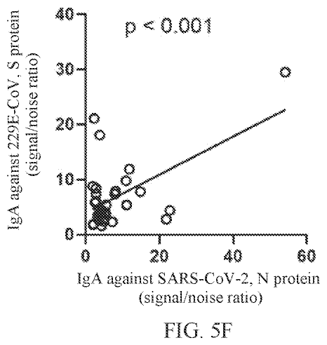

Referring to FIG. 5A to FIG. 5D, the results show, in the serum from the patients suffering from COVID-19, a positive cross-reactive correlation of the IgA against the S protein of SARS-CoV-2 with the IgGs against the S1 domain of S protein of SARS-CoV-2, the S protein of SARS-CoV, the S protein of HKU-CoV, and the S protein of OC43-CoV. Moreover, as shown in FIG. 5E and FIG. 5F, the results show, in the serum from the patients suffering from COVID-19, a positive cross-reactive correlation of the IgA against the N protein of SARS-CoV-2 with the IgGs against the N protein of SARS-CoV and the S protein of 229E-CoV.

Based on the above, the results of the detection of using the IgG or IgA in the serum from the patients suffering from COVID-19 show a positive cross-reactivity correlation of the S protein of SARS-CoV-2 with the S1 domain of S protein of SARS-CoV-2, the S protein of SARS-CoV, the S protein of HKU-CoV, the S protein of OC43-CoV, and the HA protein of influenza A H3N2 subtype. Moreover, a positive cross-reactivity correlation of the N protein of SARS-CoV-2 with the N protein of SARS-CoV and the S protein of 229E-CoV. It is demonstrated that the protein microarray with the S protein of SARS-CoV-2 and the N protein of SARS-CoV may be used to detect different types of coronaviruses and influenza viruses.

The technical solutions of the present disclosure will be described clearly and completely in combined with the drawings of the present disclosure. Obviously, the described embodiments are only one part of the embodiments of the present disclosure, but not all of the embodiments. Based on the embodiments of the present disclosure, all other embodiments obtained by a person skilled in the art without making creative efforts fall within the claim scope of the present disclosure.

Although the present disclosure has been disclosed in preferred embodiments, it is not intended to limit the present disclosure. A person having ordinary skill in the art can make various changes and modifications without departing from the concept and scope of the present disclosure. Therefore, the claimed scope of the present disclosure shall be based on the scope defined by the attached claims of the patent disclosure.

What is claimed is:

1. A protein microarray, comprising:
a substrate including a plurality of protein array blocks on a surface of the substrate; and
at least one protein immobilized on each of the plurality of protein array blocks, wherein the at least one protein is derived from a virus and comprises a spike protein and a nucleocapsid protein, the at least one protein comprises an amino acid sequence of SEQ ID NO: 1 and an amino acid sequence of SEQ ID NO: 4, and the at least one protein specifically binds to a first antibody in a to-be-tested sample or a bioagent.

2. The protein microarray according to claim 1, wherein the substrate is a glass slide or a nylon film substrate.

3. The protein microarray according to claim 1, wherein the surface of the substrate comprises an aldehyde modified layer or an amino modified layer.

4. The protein microarray according to claim 1, wherein the at least one protein further comprises a 51 domain of the spike protein, a hemagglutinin protein or a combination thereof.

5. The protein microarray according to claim 1, wherein the at least one protein further comprises an amino acid sequence selected from the group consisting of SEQ ID NO: 2, SEQ ID NO: 3, SEQ ID NO: 5, SEQ ID NO: 6, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

6. The protein microarray according to claim 4, wherein the at least one protein further comprises an amino acid sequence selected from the group consisting of SEQ ID NO: 7, SEQ ID NO: 8, SEQ ID NO: 9, SEQ ID NO: 18, SEQ ID NO: 19, and SEQ ID NO: 20.

7. The protein microarray according to claim 1, wherein the virus is selected from the group consisting of severe acute respiratory syndrome coronavirus 2 (SARS-CoV-2), middle east respiratory syndrome coronavirus (MERS-CoV), severe acute respiratory syndrome coronavirus (SARSC-CoV), human coronavirus HKU (HKU-CoV), human coronavirus 229E (229E-CoV), human coronavirus NL63 (NL63-CoV), human coronavirus OC43 (OC43-CoV), influenza A virus subtype H1N1, influenza A virus subtype H3N2, and influenza B virus.

8. The protein microarray according to claim 1, wherein the to-be-tested sample is a blood sample, a serum sample or a plasma sample from a subject.

9. The protein microarray according to claim 1, wherein the first antibody is human immunoglobulin G, human immunoglobulin A or human immunoglobulin M.

10. The protein microarray according to claim 1, wherein the bioagent is a monoclonal antibody drug or a receptor blocker.

11. The protein microarray according to claim 10, wherein the monoclonal antibody drug is a murine-derived monoclonal antibody or a rabbit-derived monoclonal antibody.

12. The protein microarray according to claim 10, wherein the monoclonal antibody drug is a monoclonal antibody against the spike protein of the SARS-CoV-2, a monoclonal antibody against the S1 domain of the spike protein of the SARS-CoV-2 or a monoclonal antibody against the nucleocapsid protein of the SARS-CoV-2.

13. The protein microarray according to claim 10, wherein the receptor blocker is a human angiotensin-converting enzyme 2 on a cell surface.

14. A method for in vitro detection of virus infection in a to-be-tested sample, comprising steps of:
providing a protein microarray according to claim 1;
adding a non-protein blocking reagent to the plurality of protein array blocks of the protein microarray, and reacting for 5 to 10 minutes to obtain a first protein microarray;
providing a to-be-tested sample from a subject, adding the to-be-tested sample to the first protein microarray, and reacting for 50 to 70 minutes followed by washing to obtain a second protein microarray;
providing a second antibody, adding the second antibody to the second protein microarray, and reacting for 25 to 35 minutes followed by washing to obtain a third protein microarray, wherein the second antibody is fluorescently labeled or enzyme-labeled; and
reading an optical signal generated from the third protein microarray by a signal reader.

15. The method according to any one of claim 14, after reading the optical signal generated from the third protein microarray by the signal reader, further comprising a step of determining whether the subject is infected by a virus, wherein the virus is selected from the group consisting of severe acute respiratory syndrome coronavirus 2 (SARS-CoV-2), middle east respiratory syndrome coronavirus (MERS-CoV), severe acute respiratory syndrome coronavirus (SARSC-CoV), human coronavirus HKU (HKU-CoV), human coronavirus 229E (229E-CoV), human coronavirus NL63 (NL63-CoV), human coronavirus OC43 (OC43-CoV), influenza A virus subtype H1N1, influenza A virus subtype H3N2, and influenza B virus.

16. A method for evaluating the ability of a bioagent to block virus infection, comprising steps of:
providing a protein microarray according to claim 1,
adding a non-protein blocking reagent to the plurality of protein array blocks of the protein microarray, and reacting for 5 to 10 minutes to obtain a fourth protein microarray;
providing a bioagent, adding the bioagent to the fourth protein microarray, and reacting for 50 to 70 minutes followed by washing to obtain a fifth protein microarray;
providing a second antibody, adding the second antibody to the fifth protein microarray, and reacting for 25 to 35 minutes followed by washing to obtain a sixth protein microarray, wherein the second antibody is fluorescently labeled or enzyme-labeled; and
reading an optical signal generated from the sixth protein microarray by a signal reader.

17. The method according to claim 16, after reading the optical signal generated from the sixth protein microarray by the signal reader, further comprising a step of evaluating the ability of the bioagent to block virus infection, wherein the virus is selected from the group consisting of severe acute respiratory syndrome coronavirus 2 (SARS-CoV-2), middle east respiratory syndrome coronavirus (MERS-CoV), severe acute respiratory syndrome coronavirus (SARSC-CoV), human coronavirus HKU (HKU-CoV), human coronavirus 229E (229E-CoV), human coronavirus NL63 (NL63-CoV), human coronavirus OC43 (OC43-CoV), influenza A virus subtype H1N1, influenza A virus subtype H3N2, and influenza B virus.

18. The method according to claim 16, wherein the bioagent is a monoclonal antibody drug or a receptor blocker.

19. The method according to claim 18, wherein the monoclonal antibody drug is a monoclonal antibody against the spike protein of the SARS-CoV-2, a monoclonal antibody against the S1 domain of the spike protein of the SARS-CoV-2 or a monoclonal antibody against the nucleocapsid protein of the SARS-CoV-2.

20. The method according to claim 18, wherein the receptor blocker is a human angiotensin-converting enzyme 2 on a cell surface.
